(19) United States
(12) Patent Application Publication
(10) Pub. No.: US 2007/0087113 A1
(43) Pub. Date: Apr. 19, 2007

(54) Surface-modified medical devices and method of making

(75) Inventors: Janelle M. Uilk, Richmond, VA (US); Joseph C. Salamone, Fairport, NY (US); Jay F. Kunzler, Canandaigua, NY (US); Daniel M. Ammon JR., Penfield, NY (US); Roya N. Borazjani, Fairport, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/254,106

(22) Filed: Oct. 19, 2005

(51) Int. Cl. A61L 33/00; B05D 3/00

(52) U.S. Cl. 427/2.1

(57) ABSTRACT

A medical device having a reduced affinity for bacterial attachment comprises a coating polymer that is attached to a surface thereof, the coating polymer providing a plurality of charges at a physiological condition. The medical device is produced by contacting the coating polymer with the medical device such that the coating polymer is attached thereto. The coating polymer may be attached to the medical device through an intermediate polymer.

### BACKGROUND OF THE INVENTION

The present invention relates to medical devices having modified surfaces and method for making such devices. In particular, the present invention relates to ophthalmic devices having surfaces modified for decreased bacterial attachment.

Advances in chemistry of materials for medical devices have increased the comfort for their extended use in a body environment. Furthermore, extended use of medical devices, such as ophthalmic lenses, has become increasingly favored due to the availability of soft contact lenses having high oxygen permeability (e.g., exhibiting high Dk values greater than 80) and/or high water content. Such lenses are increasingly made of silicone-containing materials. Although these materials have some desirable properties for ophthalmic applications, they tend to have relatively hydrophobic surfaces that have a high affinity for lipids and proteins. Accumulation of these materials can interfere with the clarity of the lens and the comfort of the wearer. In addition, hydrophobic surfaces tend to facilitate bacterial attachment thereto and growth thereon. Bacterial attachment to biomaterial surfaces is believed to be a contributing factor in device-related infection. But the extent to which a given microorganism will attach itself to a given biomaterial has proven difficult to predict. Thus, effort has been devoted to continued search for methods of preventing or reducing attachment of microorganisms to devices. U.S. Pat. No. 5,945,153 and 5,984,905 to Dearnaley disclose a method for forming an anti-bacterial coating on a surface and a medical implant having such a coating. The coating is formed by depositing a metal, such as silver, on the surface in conjunction of a layer of carbonaceous material. The metal may have low compatibility with the deposited carbonaceous material and may be released to the surrounding tissue in large amount, thereby causing possible irritation.

U.S. Pat. Nos. 5,961,958 and 5,980,868 to Homola et al. disclose a multilayered coating of antibacterial material for dental applications. The coating has a first layer of a cationic long-chain material and a second layer of a hydrophobic material, such as wax, acting as a barrier, and an anti-bacterial material being dispersed in the second layer and releasable therefrom. Due to the hydrophobicity of the barrier layer, this coating is not compatible with applications wherein the environment is hydrophilic. Furthermore, the coating is susceptible to protein deposit.

U.S. Pat. No. 6,013,106 to Tweden et al. discloses a biocompatible article having releasably adhered antimicrobial metal ions, preferably silver, which may be contained in or reversible bound to a storage structure attached to the article. Similar to other devices having antimicrobial metals, the disclosed article of this patent also presents a risk of accidental release of a concentrated amount of metal to the surrounding tissue.

U.S. Pat. No. 6,054,054 to Robertson et al. discloses a method of inhibiting the adhesion of bacterial cells to a surface of a paper machine by adding to the medium contacting the surface a cationic polymer of an organic ammonium salt. It is not expected that the cationic polymer efficiently adheres to the metal surface, and there is no teaching of a method of adhering the cationic polymer to a wide range of biocompatible surfaces. Thus, the prior art methods and devices, for one reason or another, still have shortcomings.

Therefore, there is a continued need to provide medical devices, such as ophthalmic lenses, that inhibit or have reduced affinity for bacterial attachment, and methods for making them.

### SUMMARY OF THE INVENTION

In general, the present invention provides medical devices that have reduced affinity for bacterial attachment and methods for making these devices.

In one aspect, the present invention provides a medical device having a polymer coating that is attached to a surface of the medical device. The polymer coating provides a plurality of charges at a physiological condition.

In another aspect, the coating comprises a coating polymer covalently attached to the surface of the medical device, the coating polymer having a plurality of moieties that support charges at a physiological condition. The phrase “support a charge” means generally carrying a charge by any mechanism.

In still another aspect, the coating polymer is attached to the surface of the medical device through an intermediate polymer that has functional groups capable of interacting with functional groups on the surface of the medical device and functional groups of the coating polymer.

In still another aspect, the moieties are ionizable at a physiological condition.

In still another aspect, the medical devices are ophthalmic devices.

In yet another aspect, the medical devices are contact lenses.

In a further aspect, the present invention provides a method of making a medical device that has reduced affinity for bacterial attachment. The method comprises: (a) providing the medical device having a plurality of medical-device surface functional groups; (b) providing a first polymer having a plurality of at least first-polymer functional groups capable of interacting with the medical-device surface functional groups and with at least second-polymer functional groups of a second polymer; (c) providing the second polymer having said at least second-polymer functional groups and a plurality of moieties that support a charge or are capable of becoming charged at a physiological condition; and (d) contacting the medical device with the first and second polymers at a condition sufficient to produce the medical device having reduced affinity for bacterial attachment.

Other features and advantages of the present invention will become apparent from the following detailed description and claims.

### BRIEF DESCRIPTION OF THE DRAWINGS

FIG. 1 shows the results of bacterial adherence testing of the first series of control and treated lenses.

FIG. 2 shows the results of bacterial adherence testing of the second series of control and treated lenses.

### DETAILED DESCRIPTION OF THE INVENTION

In general, the present invention provides medical devices that have reduced affinity for bacterial attachment and methods for making these devices.

In one aspect, the present invention provides a medical device having a polymer coating that is attached to a surface of the medical device. The polymer coating provides a plurality of charges at a physiological condition. The polymer coating can be attached to a surface of the medical device directly or indirectly through strong interactions such as covalent bonds or ionic interactions or otherwise through weaker interactions, such as by physical adsorption or chemisorption at the surface of the medical device.

In one aspect, the coating polymer comprises a plurality of moieties that support charges at a physiological condition, such as a condition found in an environment or on a surface of a human organ. In another aspect, such physiological condition is found in a human ocular environment, such as a condition on the human cornea. The plurality of moieties can provide negative or positive charges at a physiological condition. The coating polymer also can have a combination of some negatively charged moieties and some other positively charged moieties. In another embodiment, the coating polymer also can have moieties that are non-neutral at a physiological condition due to the presence of atoms that have unshared electrons, such as oxygen or nitrogen.

In one embodiment, the coating polymer comprises at least a polymer selected from the group consisting of polycarboxylic acids, polyamines, poly(hexamethylene biguanide), copolymers comprising alkylene oxide units, ethylenediamine with adducts of poly(alkylene oxide), 2-hydroxyethyl 2-(2-hydroxy-3-(trimethylammonio)propoxy)ethyl 2-hydroxy-3-(trimethylammonio)propyl ether cellulose (commonly known as Polyquaternium-10 or Polymer JR™), and combinations thereof.

In one aspect the polycarboxylic acids are those having free carboxylic acid moieties. In another aspect the polycarboxylic acids are selected from poly(acrylic acid), poly(methacrylic acid), copolymers thereof, copolymers comprising an alkenoic acid and acrylic acid or methacrylic acid, and combinations thereof. The carboxyl moieties of these polymers can provide negative charges at a physiological condition. In one embodiment, the alkenoic acid comprises 4 to and including 10 carbon atoms. In another embodiment, the alkenoic acid is selected from the group consisting of maleic acid, fumaric acid, itaconic acid, and combinations thereof. In still another embodiment, the coating polymer comprises poly(acrylic acid) or poly(methacrylic acid).

In another embodiment, the coating comprises: (a) polymethacrylic acid or polyacrylic acid; (b) a copolymers comprising alkylene oxide units; and (c) ethylenediamine with adducts of poly(alkylene oxide). In one aspect of this embodiment, the poly(alkylene oxide) is a block copolymer comprising poly(ethylene oxide) and poly(propylene oxide).

In still another embodiment, the coating polymer is attached to the surface of the medical device through an intermediate polymer that has functional groups capable of interacting with functional groups on the surface of the medical device and functional groups of the coating polymer. Thus, the intermediate polymer acts to couple the coating polymer to the surface of the medical device. For example, the intermediate polymer can comprise the glycidyl functional group, which is capable of forming bonds with a variety of other functional groups, such as hydroxyl, mercapto, carboxyl, or amino groups.

In yet another embodiment, the intermediate polymer comprises units of glycidyl methacrylate or glycidyl acrylate. In another embodiment, the intermediate polymer comprises poly(N,N-dimethylacrylamide-co-glycidyl methacrylate) (“poly(DMA-co-GMA)”).

In a further embodiment, the medical device has a polymer coating consisting or consisting essentially of polymethacrylic acid or polyacrylic acid. In an alternate embodiment, said polymethacrylic acid or polyacrylic acid is attached to the surface of the medical device through an intermediate polymer consisting or consisting essentially of poly(N,N-dimethylacrylamide-co-glycidyl methacrylate).

In one aspect, the medical device comprises a polymeric material and the functional groups on the surface thereof are parts of units of the polymeric material. For example, hydrogel polymers of contact lens typically comprise hydrophilic monomeric units, such as 2-hydroxyethyl methacrylate, which provides hydroxyl surface groups. Alternatively, a hydrogel polymer comprising acrylic acid or methacrylic acid units has carboxyl surface groups. In still another embodiment, a hydrogel comprising 2-aminoethyl methacrylate has amino surface groups.

In one embodiment of the present invention, wherein a contact lens comprises hydrophilic monomeric units of acrylic acid or methacrylic acid, the intermediate polymer comprises poly(glycidyl methacrylate) (“poly(GMA)”), and the coating polymer comprises poly(methacrylic acid), the surface modified contact lens is produced according to Scheme 1, wherein a, b, b₁ and b₂ are positive integers and b=b₁+b₂. The integers a, b, b₁ and b₂ can be chosen such that the coating polymer and the intermediate polymer can be easily formulated in a solvent for application to the contact lens. In one embodiment, a, b, b₁, and b₂ can range from about 10 to about 1000, or from about 50 to 500, or from about 50 to about 200, or from about 50 to about 100.

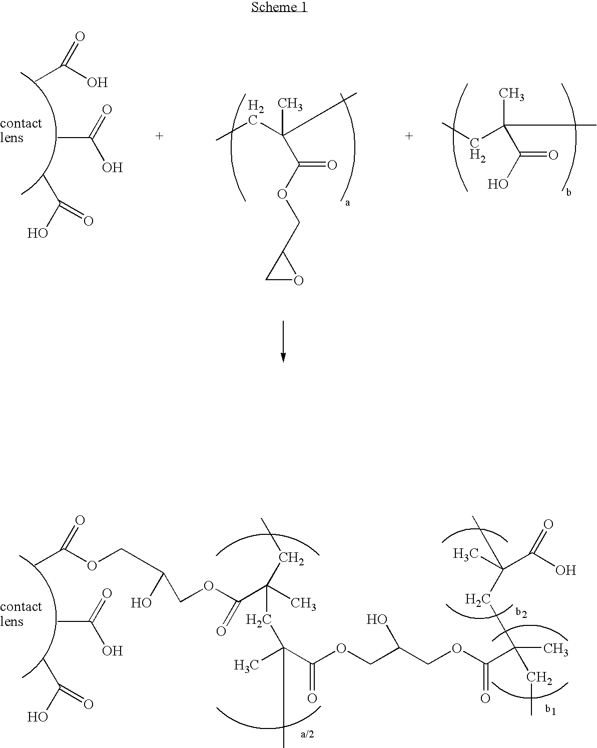

In another embodiment of the present invention, wherein a contact lens comprises hydrophilic monomeric units of acrylic acid or methacrylic acid, the intermediate polymer comprises poly(DMA-co-GMA), and the coating polymer comprises poly(methacrylic acid), the surface modified contact lens is produced according to Scheme 2, wherein u, v, x, y, b, b₁, and b₂ are positive integers, and b=b₁+b₂. The integers u, v, x, and y can be chosen such that the coating polymer and the intermediate polymer can be easily formulated in a solvent for application to the contact lens. In one embodiment, u, v, x, y, b, b₁, and b₂ can range from about 10 to about 1000, or from about 50 to 500, or from about 50 to about 200, or from about 50 to about 100.

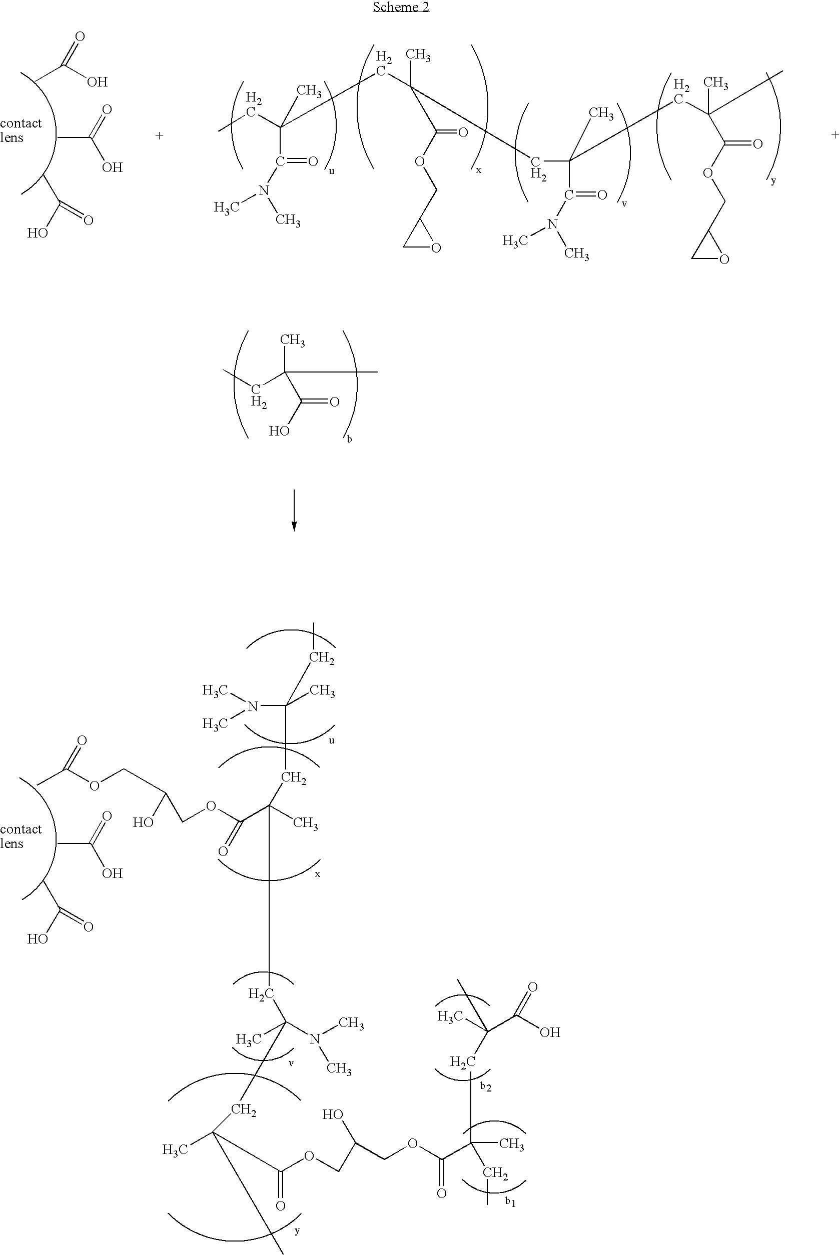

In another embodiment of the present invention, wherein a contact lens comprises hydrophilic monomeric units of acrylic acid or methacrylic acid, the intermediate polymer comprises poly(DMA-co-GMA), and the coating polymer comprises poly(methacrylic acid) and poly(ethylene oxide-propylene oxide-ethylene oxide), the surface modified contact lens is produced according to Scheme 3, wherein i, j, k, I, k₁, k₂, u, u₁, u₂, x₁, x₁, x₂, and X₃ are positive integers, k=k₁+k₂, u=u₁+u₂, and x=x₁+x₂+x₃. The integers i, j, k, I, k₁, k₂, u, u₁, u₂, x, x₁, x₂, and x₃ can be chosen such that the coating polymer and the intermediate polymer can be easily formulated in a solvent for application to the contact lens. In one embodiment, i, j, k, I, k₁, k₂, u, u₁, u₂, x, x₁, x₂, and x₃ can range from about 10 to about 1000, or from about 50 to 500, or from about 50 to about 200, or from about 50 to about 100.

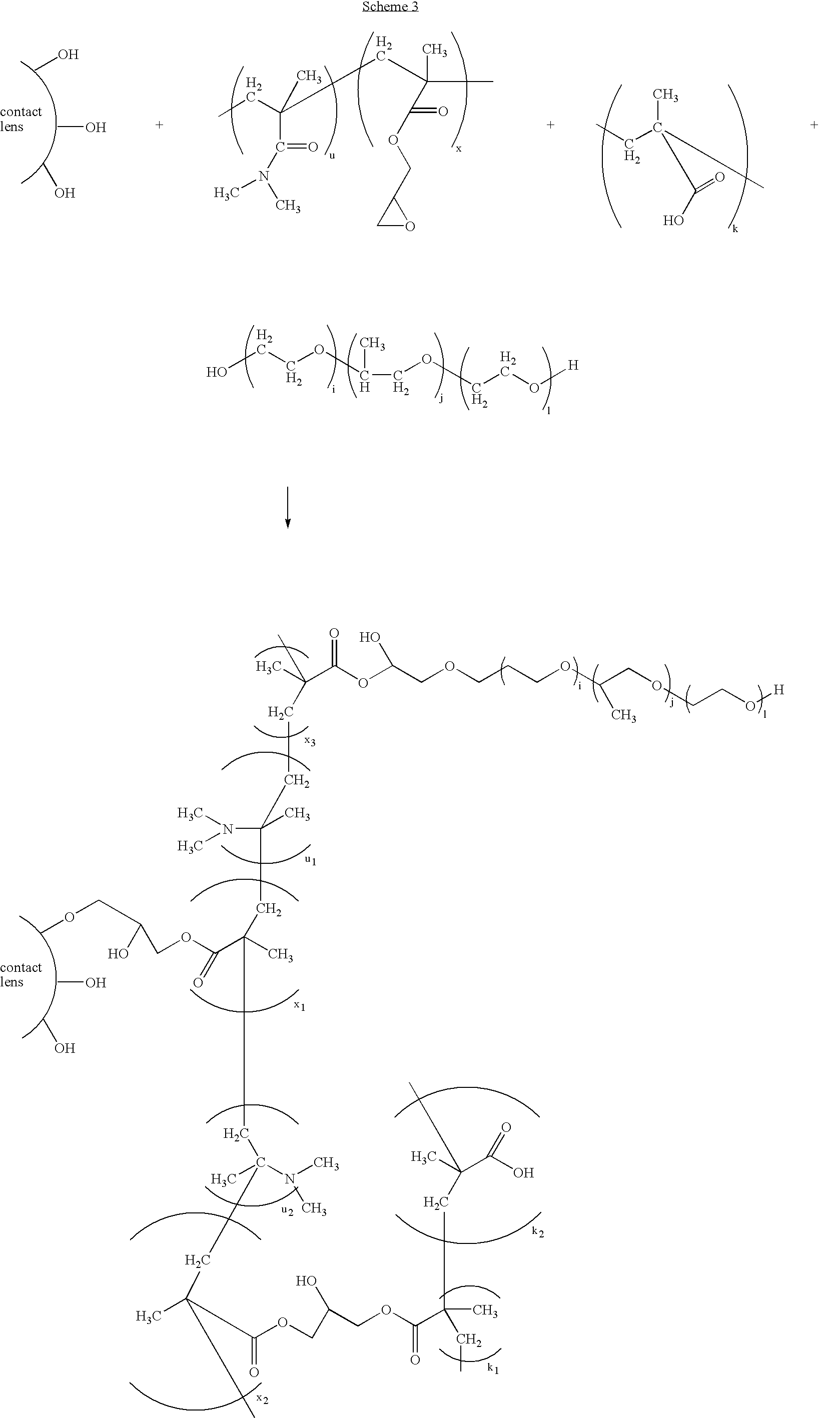

In another embodiment of the present invention, wherein a contact lens comprises hydrophilic monomeric units of 2-hydroxyethylmethacrylate (“HEMA”), the intermediate polymer comprises poly(N,N′-dimethylacrylamide-co-glycidyl methacrylate), and the coating polymer comprises poly(methacrylic acid), the surface-modified contact lens is produced according to Scheme 4, wherein u, v, x, y, k, k₁, and k₂ are positive integers, and k=k₁+k₂. The integers u, v, x, y, k, k₁, and k₂ can be chosen such that the coating polymer and the intermediate polymer can be easily formulated in a solvent for application to the contact lens. In one embodiment, u, v, x, y, k, k₁, and k₂ can range from about 10 to about 1000, or from about 50 to 500, or from about 50 to about 200, or from about 50 to about 100.

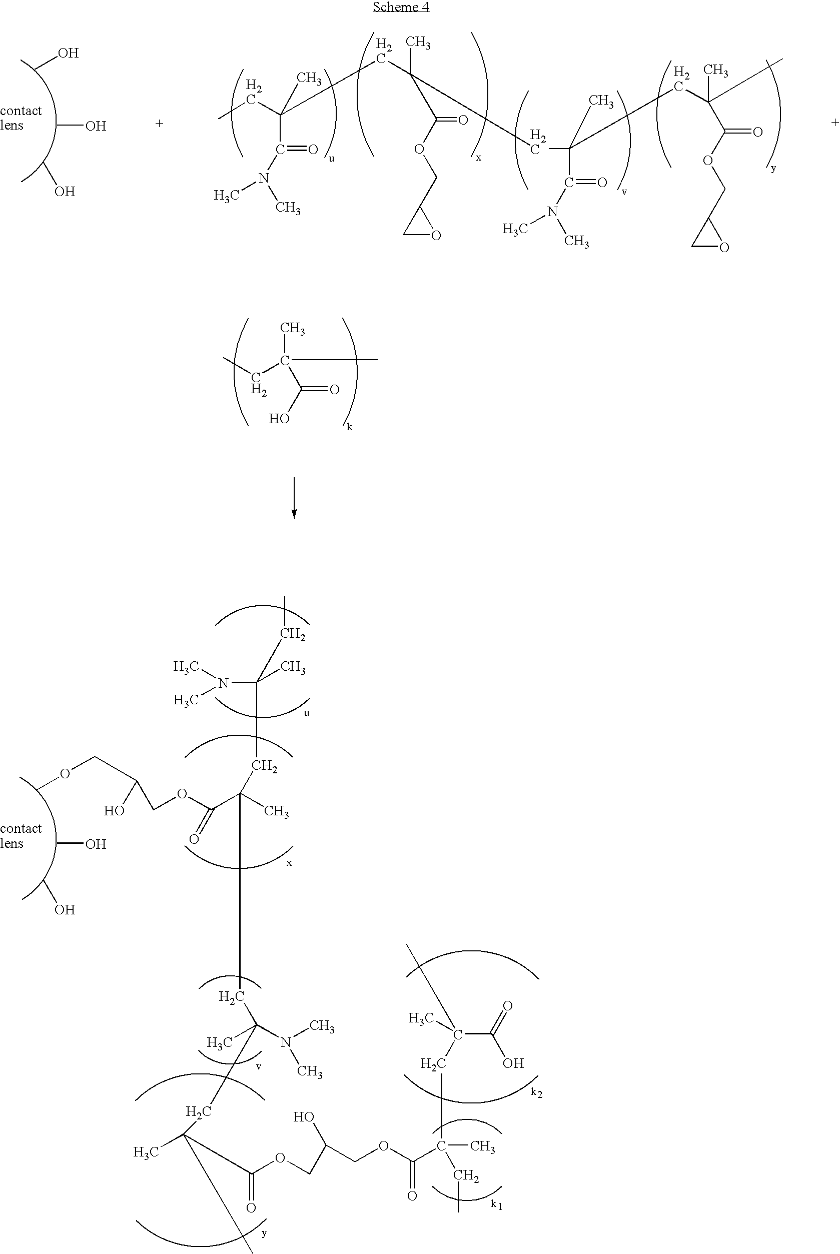

In one aspect, the surface treatment of the medical device can be carried out, for example, at about room temperature or under autoclave condition. The medical device is immersed in a solution comprising the intermediate polymer and the coating polymer. Thus, in one aspect, the medical device comes into contact with the intermediate polymer and the coating polymer substantially simultaneously. In another aspect, the medical device is immersed in a solution comprising the intermediate polymer. Then, after some elapsed time, the coating polymer is added to the solution in which the medical device is still immersed. In one embodiment of the method of treatment, the solution is aqueous.

In another aspect, the surface of the medical device can be treated with a plasma discharge or corona discharge to increase the population of reactive surface groups. The type of gas introduced into the treatment chamber is selected to provide the desired type of reactive surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate the mercaptan group. A combination of any of the foregoing gases also can be used in the treatment chamber. Methods and apparatuses for surface treatment by plasma discharge are disclosed in, for example, U.S. Pat. Nos. 6,550,915 and 6,794,456, which are incorporated herein in their entirety by reference. Such a step of treatment with a discharge can be carried out before the treated device is contacted with a medium containing the coating polymer.

Medical devices comprising a wide variety of polymeric materials, including hydrogel and non-hydrogel materials, can be made to have reduced affinity for bacterial attachment by a method of the present invention. In general, non-hydrogel materials are hydrophobic polymeric materials that do not contain water in their equilibrium state. Typical non-hydrogel materials comprise silicone acrylics, such as those formed from bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups. On the other hand, hydrogel materials comprise hydrated, cross-linked polymeric systems containing water in an equilibrium state. Hydrogel materials contain about 5 weight percent water or more (up to, for example, about 80 weight percent). Non-limiting examples of materials suitable for the manufacture of medical devices, such as contact lenses, are herein disclosed.

Hydrogel materials for medical devices, such as contact lenses, can comprise a hydrophilic monomer, such as, HEMA, methacrylic acid (“MM”), acrylic acid (“AA”), methacrylamide, acrylamide, N,N′-dimethylmethacrylamide, or N,N′-dimethylacrylamide; copolymers thereof; hydrophilic prepolymers, such as poly(alkylene oxide) having varying chain length, functionalized with polymerizable groups; and/or silicone hydrogels comprising siloxane-containing monomeric units and at least one of the aforementioned hydrophilic monomers and/or prepolymers. Hydrogel materials also can comprise a cyclic lactam, such as N-vinyl-2-pyrrolidone (“NVP”), or derivatives thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

Silicone hydrogels generally have water content greater than about 5 weight percent and more commonly between about 10 to about 80 weight percent. Such materials are usually prepared by polymerizing a mixture containing at least one siloxane-containing monomer and at least one hydrophilic monomer. Typically, either the siloxane-containing monomer or the hydrophilic monomer functions as a crosslinking agent (a crosslinking agent or crosslinker being defined as a monomer having multiple polymerizable functionalities) or a separate crosslinker may be employed. Applicable siloxane-containing monomeric units for use in the formation of silicone hydrogels are known in the art and numerous examples are provided, for example, in U.S. Pat. Nos. 4,136,250; 4,153,641; 4,740,533; 5,034,461; 5,070,215; 5,260,000; 5,310,779; and 5,358,995.

Examples of applicable siloxane-containing monomeric units include bulky polysiloxanylalkyl (meth)acrylic monomers. The term “(meth)acrylic”means methacrylic or acrylic, depending on whether the term “meth” is present or absent. An example of bulky polysiloxanylalkyl (meth)acrylic monomers are represented by the following Formula I: 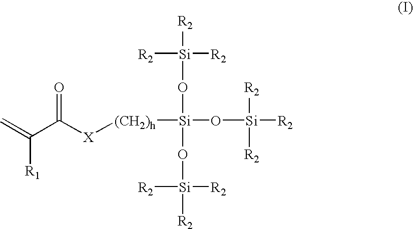
wherein X denotes —O— or —NR—; each R₁ independently denotes hydrogen or methyl; each R₂ independently denotes a lower alkyl radical, phenyl radical or a group represented by 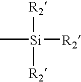
wherein each R′₂ independently denotes a lower alkyl, fluoroalkyl, or phenyl radical; and h is 1 to 10. The term “lower alkyl” means an alkyl radical having 1, 2, 3, 4, 5, 6, 7, 8, 9, or 10 carbon atoms, such as methyl, ethyl, propyl, butyl, isobutyl, pentyl, isopentyl, or hexyl radical.

A suitable bulky monomer is methacryloxypropyltris(trimethyl-siloxy)silane or tris(trimethylsiloxy)silylpropyl methacrylate (“TRIS”).

Another class of representative silicon-containing monomers includes silicone-containing vinyl carbonate or vinyl carbamate monomers such as: 1,3-bis{4-vinyloxycarbonyloxy)but-1-yl}tetramethyldisiloxane; 3-(trimethylsilyl)propyl vinyl carbonate; 3-(vinyloxycarbonylthio)propyl-{tris(trimethylsiloxy)silane}; 3-{tris(trimethylsiloxy)silyl}propyl vinyl carbamate; 3-{tris(trimethylsiloxy)silyl} propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

Another class of representative silicon-containing monomers includes silicone-containing vinyl carbonate or vinyl carbamate monomers such as: 1,3-bis {4-vinyloxycarbonyloxy)but-1-yl}tetramethyl-disiloxane; 3-(trimethylsilyl )propyl vinyl carbonate; 3-(vinyloxycarbonylthio)propyl-{tris(trimethylsiloxy)silane}; 3-{tris(tri-methylsiloxy)silyl} propyl vinyl carbamate; 3-{tris(trimethylsiloxy)silyl} propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II: 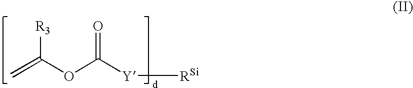
wherein:
Y′denotes —O—, —S— or —NH—;
R^{Si} denotes a silicon-containing organic radical;
R₃ denotes hydrogen or methyl; and
d is 1, 2, 3 or 4.

Suitable silicon-containing organic radicals R^{Si} include the following: 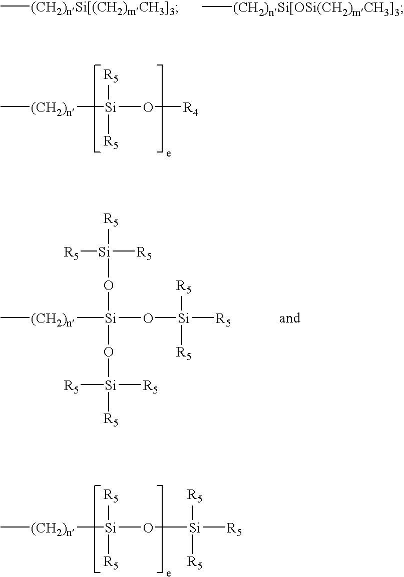
wherein
R₄ denotes 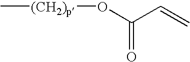
   wherein p′ is from 1 to and including 6;
R₅ denotes an alkyl radical or a fluoroalkyl radical having from 1 to and including 6 carbon atoms;
e is 1 to 200; n′ is 1, 2, 3 or 4; and m′ is 0, 1, 2, 3, 4 or 5.

An example of a particular species within Formula II is represented by Formula III.

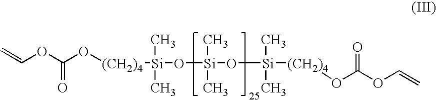

Another class of silicon-containing monomer includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacrylates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:
E(*D*A*D*G)ₐ*D*A*D*E′ (IV)
or
E(*D*G*D*A)ₐ*D*G*D*E′ (V),
wherein:
D denotes an alkyl diradical, an alkyl cycloalkyl diradical, a cycloalkyl diradical, an aryl diradical or an alkylaryl diradical having 6 to 30 carbon atoms;
G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;
* denotes a urethane or ureylene linkage;
a is at least 1;
A denotes a divalent polymeric radical of Formula VI: 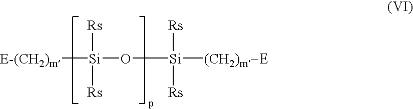
   wherein:
each Rₛ independently denotes an alkyl or fluoro-substituted alkyl group having 1 to 10 carbon atoms which may contain ether linkages between carbon atoms;
m′ is at least 1; and
p is a number which provides a moiety weight of 400 to 10,000;
each of E and E′ independently denotes a polymerizable unsaturated organic radical represented by Formula VII:
   wherein:
R₆ is hydrogen or methyl;
R₇ is hydrogen, an alkyl radical having from 1 to and including 6 carbon atoms, or a —CO—Y—R₉ radical wherein Y is —O—, —S— or —NH—;
R₈ is a divalent alkylene radical having from 1 to and including 10 carbon atoms;
R₉ is a alkyl radical having from 1 to and including 12 carbon atoms;
X denotes —CO— or —OCO—;
Z denotes —O— or —NH—;
Ar denotes a substituted or unsubstituted aromatic radical having from 6 to and including 30 carbon atoms;
w is from 0 to and including 6; x is 0 or 1; y is 0 or 1; and z is 0 or 1.

A more specific example of a silicone-containing urethane monomer is represented by Formula VIII: 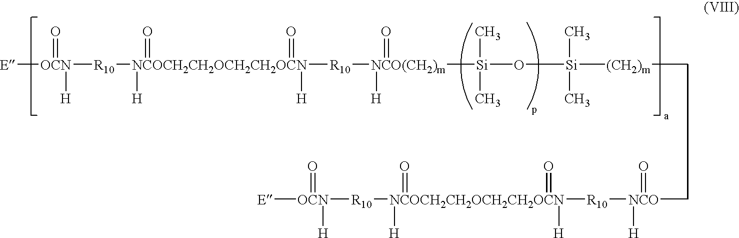
wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

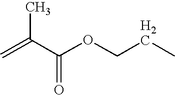

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more poly(siloxanylalkyl (meth)acrylic) monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

In particular regard to contact lenses, the fluorination of certain monomers used in the formation of silicone hydrogels has been indicated to reduce the accumulation of deposits on contact lenses made therefrom, as described in U.S. Pat. Nos. 4,954,587, 5,079,319 and 5,010,141. Moreover, the use of silicone-containing monomers having certain fluorinated side groups (e.g., —(CF₂)—H) have been found to improve compatibility between the hydrophilic and silicone-containing monomeric units, as described in U.S. Pat. Nos. 5,387,662 and 5,321,108.

In another aspect, a polymeric material of the present invention comprises an additional monomer selected from the group consisting of hydrophilic monomers and hydrophobic monomers.

Hydrophilic monomers can be nonionic monomers, such as 2-hydroxyethyl methacrylate (“HEMA”), 2-hydroxyethyl acrylate (“HEA”), 2-(2-ethoxyethoxy)ethyl (meth)acrylate, glyceryl (meth)acrylate, poly(ethylene glycol (meth)acrylate), tetrahydrofurfuryl (meth)acrylate, (meth )acrylamide, N,N′-dimethylmethacrylamide, N,N′-dimethylacrylamide(“DMA”), N-vinyl-2-pyrrolidone (or other N-vinyl lactams), N-vinyl acetamide, and combinations thereof. Other hydrophilic monomers can have more than one polymerizable group, such as tetraethylene glycol (meth)acrylate, triethylene glycol (meth)acrylate, tripropylene glycol (meth)acrylate, ethoxylated bisphenol-A (meth)acrylate, pentaerythritol (meth)acrylate, pentaerythritol (meth)acrylate, ditrimethylolpropane (meth)acrylate, ethoxylated trimethylolpropane (meth)acrylate, dipentaerythritol (meth)acrylate, alkoxylated glyceryl (meth)acrylate. The term “(meth)acrylate” means methacrylate or acrylate. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process. Non-limiting examples of such substituted anionic hydrophilic monomers include trimethylsilyl esters of (meth)acrylic acid, which are hydrolyzed to regenerate an anionic carboxyl group. The hydrophilic monomer also can be a cationic monomer selected from the group consisting of 3-methacrylamidopropyl-N,N,N-trimethylammonium salts, 2-methacryloyloxyethyl-N,N,N-trimethylammonium salts, and amine-containing monomers, such as 3-methacrylamidopropyl-N,N-dimethyl amine. Other suitable hydrophilic monomers will be apparent to one skilled in the art.

Non-limiting examples of hydrophobic monomers are C₁-C₂₀ alkyl and C₃-C₂₀ cycloalkyl (meth)acrylates, substituted and unsubstituted aryl (meth)acrylates (wherein the aryl group comprises 6 to 36 carbon atoms), (meth)acrylonitrile, styrene, lower alkyl styrene, lower alkyl vinyl ethers, and C₂-C₁₀ perfluoroalkyl (meth)acrylates and correspondingly partially fluorinated (meth)acrylates.

Solvents useful in the surface treatment of the medical device, such as a contact lens, include solvents that readily solubilize the polymers such as water, alcohols, lactams, amides, cyclic ethers, linear ethers, carboxylic acids, and combinations thereof. Preferred solvents include tetrahydrofuran (“THF”), acetonitrile, N,N-dimethyl formamide (“DMF”), and water. The most preferred solvent is water.

Surface Treatment of Contact Lenses for Reduction of Bacterial Attachment

Materials: SofLens59™ contact lenses (a hydrogel type of contact lenses, available from Bausch & Lomb Incorporated, Rochester, N.Y.) were coated to produce contact lenses having reduced bacterial attachment. Poly(DMA-co-GMA) was synthesized in house, having 86 mole percent DMA and 14 mole percent GMA, by polymerizing DMA and GMA in the presence of a radical polymerization initiator, such as 2,2′-azobisisobutyronitrile. Such a polymerization is within the skill of the person skilled in the art. Solutions of poly(DMA-co-GMA) and poly(acrylic acid) polymers were made up by adding purified water to a known amount of polymer and stirred in a covered container for 50-65 minutes to ensure complete dissolution of the polymer. Solution of Polymer JR™ required heating of the solution to about 60° C. for at least one hour to ensure dissolution of the polymer. Polymer JR™ is available commercially from Amerchol (Edison, N.J.). The solution of poly(hexamethylene biguanide) (“PHMB”) was prepared by dilution of 20% (by weight) PHMB solution to 8% (by weight) or less PHMB in solution using water.

A general method of coating is now described. Medical devices, such as commercial SofLens59™ contact lenses, were removed from the packaging and soaked in purified water for at least 15 minutes prior to being placed in polymer solution. It should be recognized by persons skilled in the art that the quantities of a solution disclosed herein may be adjusted under specific circumstances to accommodate the size of the medical device. Glass vials were labeled and filled with about 4 ml of a polymer solution, and a lens is placed in each vial. When two polymer solutions were used for coating, they were mixed together immediately prior to placing in the vials. The vials were capped with silicone stoppers and crimped aluminum caps, then placed in an autoclave for one 30-minute cycle. The treated lenses were allowed to cool for a minimum of 3 hours, then removed from the vials and rinsed at least three times with deionized water. The rinsed lenses were then placed into new vials containing 4 ml of borate buffered saline (phosphate for samples undergoing bacterial adhesion testing) and autoclaved for one 30-minute cycle for sterilization. Samples treated with PHMB were rinsed three times with 1 N HCI solution to remove any loosely bound PHMB from the lenses, then rinsed with deionized water before being placed in the saline solution. The various designations of untreated (control) and treated lenses are explained in Table 1.

TABLE 1

| Designation | Treatment |
|---|---|
| Soflens59 | untreated Soflens59 ™ contact lenses, control sample |
| Soflens5 + 7 | Soflens59 ™ contact lenses treated with solution 7⁽¹⁾ |
| DMA-co-GMA | treated with 0.5% solution containing poly(DMA-co-GMA) |
| DMA-co-GMA + 7 | treated with 0.5% solution containing poly(DMA-co-GMA) |
| | and solution 7 |
| DMA-co- | treated with 0.5% solution containing poly(DMA-co-GMA) and |
| GMA + PJR | 0.1% Polymer JR ™ |
| DMA-co- | treated with 0.5% solution containing poly(DMA-co-GMA), |
| GMA + PJR + 7 | 0.1% Polymer JR ™ and solution 7 |
| DMA-co- | treated with 0.5% solution containing poly(DMA-co-GMA) |
| GMA + PAA | and 0.1% poly(acrylic acid) |
| DMA-co- | treated with 0.5% solution containing poly(DMA-co-GMA), 0.1% |
| GMA + PAA + 7 | poly(acrylic acid) and solution 7 |
| DMA-co- | treated with poly(hexamethylene biguanide) |
| GMA + PHMB | |
| DMA-co- | treated with poly(hexamethylene biguanide) and solution 7 |
| GMA + PHMB + 7 | |

Notes:
⁽¹⁾Solution 7 is a buffer solution comprising boric acid (0.85%), sodium phosphate (0.46%), hydroxyalkylphosphonate (0.03%), Tetronic 1107 ™ (a ethylene diamine surfactant having four poly(propylene oxide-ethylene oxide) adducts, available from BASF, 1%), Pluronic F-127 ™ (a copolymer of poly(ethylene oxide-propylene oxide-ethylene oxide), available from BASF, 2%), and Polymer JR ™ (0.02%); all compositions in weight percent.

Control and treated lenses were tested for adherence of Pseudomonas aeruginosa bacteria using a modification of the procedures disclosed by Sawant et al., Curr. Microbiol., Vol. 22, 285-292(1991), and Ahearn et al., Methods in Enzymology, Vol. 310, 551-557 (1999). Bacterial cells were grown in Triptic Soy Broth (“TSB”) at 37° C. on a rotary shaker for 12 to 18 hours. Cells were harvested by centrifugation at 3000×g for 10 min, washed two times in 0.9% saline and suspended in minimal medium(1.0 g D-glucose, 7.0 g K₂HPO₄, 2.0 g KH₂PO₄, 0.5 g sodium citrate, 1.0 g (NH₄)₂SO₄, and 0.1 g MgSO₄ in 1 liter distilled H₂O, pH 7.2) to a concentration of about ˜2×10⁸ cells per ml (Optical Density 0.10 at 600 nm). The minimal broth cultures were incubated for 1 hour at 37° C. with shaking. One to 3 μCi/ml of L-[3,4,5-³H] leucine (NEN Research Products, Du Pont Company, Wilmington, Del.) were added to the cells and the cell suspensions were incubated for another 20 minutes. These cells were washed 4 times in 0.9% saline and suspended in phosphate buffered saline (PBS) to a concentration of about ˜10⁸ cells per ml (Optical Density 0.10 at 600 nm).

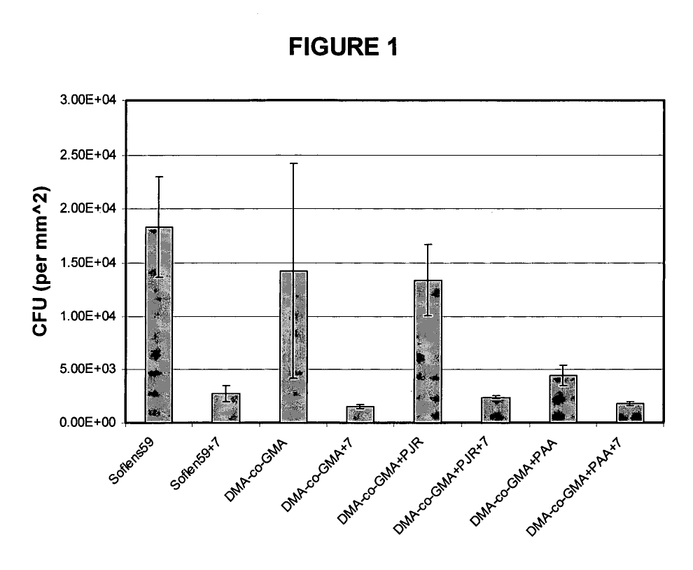

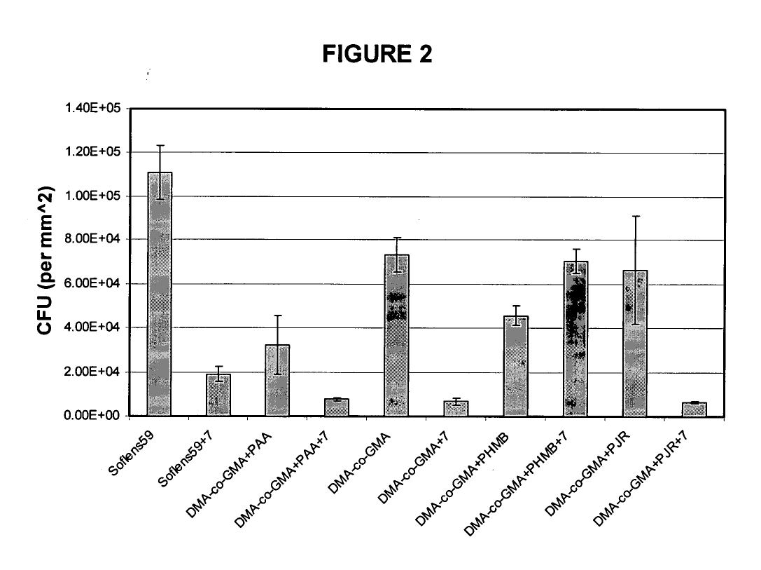

Control and treated lenses, as described above, were incubated with 3 ml of the radiolabeled cell suspension at 37° C. for 2 hours. These lenses were removed from the cell suspension with a sterile forceps and immersed 5 times in each of three successive aliquots (180 ml) of initially sterile 0.9% saline solution. The lenses were shaken free from saline and transferred to 20-ml glass scintillation vials. Ten milliliters of Opti-Fluor scintillation cocktail (Packard Instrument Co., Downers Grove, Ill.) were added to each vial. The vials were vortexed and then placed in a liquid scintillation counter (LS-7500, Beckman Instruments, Inc., Fullerton, Calif.). Data for two experiments were converted from disintegrations per min (“dpm”) to colony-forming units (“cfu”) based on a standard calibration curve and expressed as cfu/mm². Calibration curves were constructed from numbers of colonies recovered in pour plates of serial dilutions of inocula and from optical densities (“O.D.s”) of serial dilutions of cell suspensions of known densities. Results of the testing are shown in FIGS. 1 and 2 for two series of tests, each with its control samples. These results show that a coating comprising a coating polymer capable of supporting charges such as a polycarboxylic acid (e.g., poly(acrylic acid) or poly(methacrylic acid)) or a polymer comprising a plurality of poly(ethylene oxide-polypropylene oxide-ethylene oxide) units can substantially inhibit attachment of bacteria to the lenses. In addition, a further reduction of bacteria attachment is realized when such a lens also includes an intermediate polymer such as poly(DMA-co-GMA).

Other types of contact lenses, such as those comprising other hydrogel materials can be treated with coating polymers, as disclosed above. In one embodiment, PureVision™ contact lenses comprising Balafilcon A hydrogel material, disclosed in U.S. Pat. No. 5,260,000, which is incorporated herein by reference, were surface-treated with a coating polymer as disclosed above. (PureVision™ contact lenses are available from Bausch and Lomb Incorporated, Rochester, N.Y.) In one aspect, PureVision™ contact lenses were first treated with a plasma discharge generated in a chamber containing air and ammonia to increase the population of reactive surface functional groups. The solution for surface treatment comprised poly(DMA-co-GMA) and poly(acrylic acid).

The present invention also provides a method for producing a medical device having a reduced affinity for attachment of bacteria. In one aspect, the method comprises: (a) providing the medical device having a plurality of medical-device surface functional groups; (b) providing a first polymer having a plurality of at least first-polymer functional groups capable of interacting with the medical-device surface functional groups and with at least second-polymer functional groups of a second polymer; (c) providing the second polymer having said at least second-polymer functional groups and a plurality of moieties that support a charge or are capable of becoming charged at a physiological condition; and (d) contacting the medical device with the first and second polymers at a condition sufficient to produce the medical device having reduced affinity for bacterial attachment. In one aspect, the medical device is contacted with the first and the second polymers substantially simultaneously. In another aspect, the medical device may be contacted with the first polymer in a medium. The second polymer is subsequently added into the medium after an elapsed time to produce the finally treated medical device.

The step of contacting can be effected at ambient condition or under autoclave condition. The temperature for treatment can range from ambient to about 100° C., or from slightly above ambient temperature to about 80° C. The treatment time can range from about 10 seconds to about 48 hours, or from about 1 minute to about 24 hours, or from about 10 minutes to about 4 hours, or from about 10 minutes to about 2 hours.

In another aspect, the method further comprises the step of treating the surface of the medical device to increase a population of the medical-device surface functional groups before the step of contacting the medical device with the first and second polymers. In still another aspect, the step of treating the surface of the medical device is carried out in a plasma discharge or corona discharge environment. In yet another aspect, a gas is supplied to the discharge environment to provide the desired surface functional groups.

Medical devices having a coating of the present invention can be used advantageously in many medical procedures. For example, contact lenses having a coating and/or produced by a method of the present invention can be advantageously used to correct the vision of the natural eye.

Medical articles that are in contact with body fluid, such as a wound dressing, catheters, implants (e.g., artificial hearts or other artificial organs), can be provided with a coating of the present invention to reduce bacterial attachment and growth thereon.

In a further aspect, the present invention provides a method of making a medical device that has reduced affinity for bacterial attachment. The method comprises: (a) forming the medical device comprising a polymeric material having a plurality of medical-device surface functional groups; (b) providing a first polymer having a plurality of at least first-polymer functional groups capable of interacting with the medical-device surface functional groups and with at least second-polymer functional groups of a second polymer; (c) providing the second polymer having said at least second-polymer functional groups and a plurality of moieties that support a charge or are capable of becoming charged at a physiological condition; and (d) contacting the medical device with the first and second polymers at a condition sufficient to produce the medical device having reduced affinity for bacterial attachment.

Non-limiting examples of materials for the medical device and the first and the second polymers are disclosed above.

In one embodiment, the medical device is formed by disposing precursors for the medical device material in a cavity of a mold, which cavity has the shape of the medical device, and polymerizing the precursors.

In another embodiment, a solid block of a polymeric material is first produced, then the medical device is formed from such a solid block; e.g., by shaping, cutting, lathing, machining, or a combination thereof.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

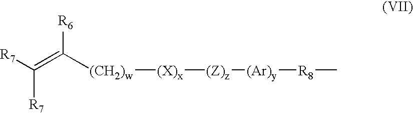

What is claimed is:

1. A medical device comprising a coating polymer that is attached to a surface thereof, the coating polymer providing a plurality of charges at a physiological condition.

2. The medical device of claim 1, wherein the coating polymer is attached to the surface of the medical device through an intermediate polymer that has functional groups capable of interacting with functional groups of the surface of the medical device and functional groups of the coating polymer.

3. The medical device of claim 1, wherein the plurality of charges are selected from negative charges, positive charges, and combinations thereof.

4. The medical device of claim 1, wherein the plurality of charges are negative charges.

5. The medical device of claim 2, wherein the coating polymer comprises a poly(carboxylic acid).

6. The medical device of claim 5, wherein the poly(carboxylic acid) is selected from the group consisting of poly(acrylic acid), poly(methacrylic acid), and copolymers thereof, copolymers of an alkenoic acid and acrylic acid, copolymers of an alkenoic acid and methacrylic acid, and combinations thereof.

7. The medical device of claim 5, wherein the coating polymer further comprises hydrophilic monomeric units.

8. The medical device of claim 7, wherein the hydrophilic units are selected from the group consisting of neutral monomers, cationic monomers, ampholytic monomers, and combinations thereof.

9. The medical device of claim 8, wherein the neutral monomers are selected from the group consisting of N,N-dimethylacrylamide, N-vinylpyrrolidone, (meth)acrylamide, 2-hydroxyethyl methacrylate, glyceryl methacrylate, and combinations thereof.

10. The medical device of claim 5, wherein the coating polymer further comprises a polymeric material comprising poly(alkylene oxide) units.

11. The medical device of claim 2, wherein the coating polymer consists of a poly(carboxylic acid).

12. The medical device of claim 2, wherein the coating polymer comprises poly(hexamethylene biguanide).

13. The medical device of claim 2, wherein the coating polymer is attached to the intermediate polymer by covalent bonds.

14. The medical device of claim 1, wherein the medical device comprises a polysiloxane.

15. The medical device of claim 1, wherein the medical device is an ophthalmic device.

16. The medical of claim 1, wherein the medical device is a contact lens.

17. A medical device comprising a coating polymer that is attached to a surface thereof through an intermediate polymer, the coating polymer providing a plurality of charges at a physiological condition and comprising a poly(carboxylic acid) and poly(alkylene oxide) units; wherein the intermediate polymer comprises poly(DMA-co-GMA), and the poly(carboxylic acid) is selected from the group consisting of poly(acrylic acid), poly(methacrylic acid), combinations thereof, and copolymers thereof.

18. The medical device of claim 17, wherein the coating polymer is covalently attached to the surface of the medical device through the intermediate polymer.

19. A method for reducing bacterial attachment to a medical device, the method comprising:
(a) providing the medical device having a plurality of medical-device surface functional groups;
(b) providing a first polymer having a plurality of at least first-polymer functional groups capable of interacting with the medical-device surface functional groups and with at least second-polymer functional groups of a second polymer;
(c) providing the second polymer having said at least second-polymer functional groups and a plurality of moieties that support a charge or are capable of becoming charged at a physiological condition; and
(d) contacting the medical device with the first and second polymers at a condition sufficient to produce the medical device having reduced affinity for bacterial attachment.

20. The method of claim 19, further comprising the step of increasing a population of the medical-device surface functional groups before the step of contacting.

21. The method of claim 20, wherein the step of increasing the population of the medical-device surface functional groups is carried out in a plasma discharge or a corona discharge environment.

22. The method of claim 20, wherein the step of increasing the population of the medical-device surface functional groups is effected by a plasma discharge treatment.

23. The method of claim 19, wherein the second polymer comprises a poly(carboxylic acid).

24. The method of claims 23, wherein the poly(carboxylic acid) is selected from the group consisting of poly(acrylic acid), poly(methacrylic acid), and copolymers thereof, copolymers of an alkenoic acid and acrylic acid, copolymers of an alkenoic acid and methacrylic acid, and combinations thereof.

25. The method of claim 24, wherein the second polymer further comprises a polymeric material comprising poly(alkylene oxide) units.

26. The method of claim 19, wherein the medical device is contacted with the first and second polymers substantially simultaneously.

27. A method for making a medical device, the method comprising:
(a) forming the medical device comprising a polymeric material having a plurality of medical-device surface functional groups;
(b) providing a first polymer having a plurality of at least first-polymer functional groups capable of interacting with the medical-device surface functional groups and with at least second-polymer functional groups of a second polymer;
(c) providing the second polymer having said at least second-polymer functional groups and a plurality of moieties that support a charge or are capable of becoming charged at a physiological condition; and
(d) contacting the medical device with the first and second polymers at a condition sufficient to produce the medical device having reduced affinity for bacterial attachment.

28. The method of claim 27, further comprising the step of increasing a population of the medical-device surface functional groups before the step of contacting.

29. The method of claim 28, wherein the step of increasing the population of the medical-device surface functional groups is carried out in a plasma discharge or a corona discharge environment.

30. The method of claim 27, wherein the step of forming comprising shaping the medical device from a polymeric material.
